(19) United States
(12) Patent Application Publication
(10) Pub. No.: US 2026/0358336 A1
(43) Pub. Date: Nov. 12, 2026

(54) METHOD TO DETERMINE EXTRACELLULAR VESICLE RECOVERY

(71) Applicants: EXIMMIUM BIOTECHNOLOGIES GMBH, München (DE); MICROCOAT BIOTECHNOLOGIE GMBH, Bernried am Starnberger See (DE)

(72) Inventors: Kathrin GÄRTNER, München (DE); Sonja MOLINARO, Weilheim (DE); Marc JURASCHITZ, Murnau a. Staffelsee (DE)

(73) Assignees: EXIMMIUM BIOTECHNOLOGIES GMBH, München (DE); MICROCOAT BIOTECHNOLOGIE GMBH, Bernried am Starnberger See (DE)

(21) Appl. No.: 19/123,726

(22) PCT Filed: Oct. 26, 2023
(86) PCT No.: PCT/EP2023/079944

§ 371 Date: Apr. 23, 2025

(30) Foreign Application Priority Data
Oct. 26, 2022 (EP) 22203729.3

(51) Int. Cl. G01N 33/569; C12N 5/071; G01N 33/58

(52) U.S. Cl. CPC G01N 33/56966; C12N 5/0686; G01N 33/581; G01N 2333/908

(57) ABSTRACT

The present invention relates to a method to determine the recovery rate of sample extracellular vesicles. The present invention further relates to a kit comprising a control extracellular vesicle. The present invention also relates to methods for preparing control extracellular vesicles, a method to calibrate a device for sample extracellular vesicles analysis, and a method to evaluate the isolation of extracellular vesicles.

### CROSS-REFERENCE TO RELATED APPLICATIONS

The present application claims the benefit of priority of EP Patent Application No. 22 203 729.3 filed 26 Oct. 2022, the content of which is hereby incorporated by reference in its entirety for all purposes.

The present invention relates to a method to determine the recovery rate of sample extracellular vesicles. The present invention further relates to a kit comprising a control extracellular vesicle. The present invention also relates to methods for preparing control extracellular vesicles, a method to calibrate a device for sample extracellular vesicles analysis, and a method to evaluate the isolation of extracellular vesicles.

Extracellular vesicles (EVs) are nanometer-sized vesicles secreted by all cell types and found circulating in body fluids such as plasma, serum, urine, cerebrospinal fluid, etc. EVs consist of a double lipid membrane surrounding its cargo of proteins, nucleic acids and metabolites making them useful conveyors of information as the lipid membrane provides protection against degradative proteins, such as proteases and RNAses, present in biofluids.

EVs are found to be implicated in, e.g. cancer progression. They can be secreted by cancer cells and either activate or recruit cancer or stromal cells locally or educate a premetastatic niche at a distant location, and hereby facilitating metastasis formation. EVs isolated from different biofluids have been found to contain specific mRNA, miRNA and protein content related to disease state for breast, urogenital, pancreatic and ovarian cancers (Sadovska et al., Anticancer Res (2015), 35:6379-6390; Nawaz et al., Nat Rev Urol (2014), 11:688-701, Tang et al., Cancer Lett (2015), 367:26-33, Melo et al., Nature (2015), 523:177-182). However, little advances are made towards clinical application. This is mainly due to the fact that EV research is difficult to reproduce owing to the plethora of isolation methods used and a lack of standardization and normalization (Lotvall et al., J Extracell Vesicles (2014), 3:1-6, Van Dein et al., Nat Methods (2017), 14:228-232).

Up to date there are few materials used as a reference material for EV research. Nanometer sized polystyrene or silica beads are often used as a calibrator for flow cytometry (FC) or nanoparticle tracking analysis (NTA). Yet, this usually leads to inaccurate measurements of EVs. Another alternative could be EV-inspired liposomes engineered to contain proteins and nucleic acids. However, natural EVs are heterogenous in size, and standards lacking this heterogeneity may consequently impact the standard reference settings of EV quantification methods.

Alternatively, physical methods used to determine the concentration of EVs and in particular nanotracking analysis (NTA, Zetaview, Malvern, etc.) are common in the field. However, these methods cannot address the central problem of analyzing complex biological or medical samples for their content and concentration of EVs. This is because physical methods fail in samples with very high protein concentration such as serum or plasma probes. As a consequence, the samples need to be processed and further purified prior to their analysis but nobody knows the quantitative loss of EVs during the downstream process of sample preparation and purification. Protocols and reference EVs are lacking.

Similarly, detection of EVs using antibodies directed against paradigmatic EV marker proteins is hampered by the fact that quantitative parameters do not exist. As a consequence, (lower) limits of detection of EVs are unknown which is a major problem in this field of in vitro diagnostics (IVD). With currently established protocols direct or indirect quantitation of EVs with the aid of antibodies is impossible because reference EVs with known and certified composition and concentration are not available for academia or industry. Moreover, limits of detections are also unknown when it comes to subfractions of EVs some of which are probably organ-specific and could be a potential source of new diagnostic approaches.

US20130078658 discloses a recombinant EV comprising a fusion of a type I or II membrane protein and a fluorescent protein (providing for biofluorescence) or luciferase (providing for chemiluminescence) which can be used to monitor sample EVs. However, the latter recombinant EVs are difficult to produce in high quantities and cannot be detected in a very sensitive manner. WO2019091964 discloses EVs with fusions of Gag and fluorescent proteins such as eGFP located intraluminally, i.e., inside of the EV. However, this approach requires time intense assays (e.g., fNTA, ELISA-mediated detection of Gag protein after EV lysis, etc.) and is not suitable for high-throughput applications.

These and further disadvantages need to be overcome. The present invention therefore addresses these needs and technical objectives and provides a solution as described herein and as defined in the claims. The present invention provides several methods that address these difficulties using exogenous standards to be used as reference EVs.

The present invention relates to a method to determine the recovery rate of sample extracellular vesicles, the method comprising:
(a) mixing a biofluid sample comprising sample extracellular vesicles with a known amount of control extracellular vesicles, wherein the control extracellular vesicles comprise
   a heterologous marker wherein said marker is located (i) on the outer side or (ii) on the inner side of the control extracellular vesicle (preferably on the outer side) and which is suitable for colorimetric, photometric, and/or fluorescent detection (preferably colorimetric or photometric detection),
   to obtain a mixture,
(b) isolating the extracellular vesicles from said mixture,
(c) detecting the amount of control extracellular vesicles among the isolated extracellular vesicles using
   (c1) a colorimetric photometric, or fluorescent detection assay (preferably a colorimetric or photometric detection assay), or
   (c2) an immuno-based assay (e.g., ELISA, EIA, Western Blot, ELISpot, and other suitable immune-based assays as known in the art) employing a binding agent as described herein which (specifically) binds to said heterologous marker, wherein said marker is located on the outer side, and
(d) determining the recovery rate of said sample extracellular vesicles based on the ratio of the amount of control extracellular vesicles detected in step (c) after the isolation step to the known amount of control extracellular vesicles mixed with the biofluid sample prior to the isolation step.

In context with the present invention, employing step (c1) is preferred for detecting the amount of control extracellular vesicles among the isolated extracellular vesicles according to step (c) of the method described and provided herein.

As used herein, unless specified otherwise, the term “amount” in context with extracellular vesicles means the number of vesicles.

In one embodiment of the present invention, the binding agent employed in step (c2) of the method described and provided herein may further comprise a heterologous marker (e.g., HRP) as described herein.

In another embodiment of the present invention, as step (c2′), alternatively to employing a binding agent (specifically) binding to said heterologous marker according to step (c2), the control extracellular vesicles may be PEGylated (i.e. covalently or non-covalently linked to polyethylene glycol (PEG)) and then an immune-based assay (e.g., ELISA, EIA, Western Blot, ELISpot, and other suitable immune-based assays as known in the art) employing a binding agent as described herein which (specifically) binds to PEG is used. This approach may also be used to separate the control extracellular vesicles from the sample extracellular vesicles.

As used herein, the term “sample extracellular vesicle (sample EV)” refers to endogenous EVs present in a biofluid sample as described herein. Such sample EVs are already contained in a biofluid sample or are suspected to be contained in a biofluid sample.

As used herein, the term “control extracellular vesicles (control EV)” refers to EVs which are not a priori present in a biofluid sample as described herein, but which are exogenously or “extra” added, e.g. mixed with sample EVs contained or suspected to be contained in a biofluid sample. Control EVs may; e.g. be obtainable from production cell line which is, e.g. transfected or transduced with corresponding by transfecting an appropriate host cell with corresponding constructs, e.g. an expression vector.

As has been surprisingly found in context with the present invention, extracellular vesicles with a heterologous marker suitable for colorimetric, photometric, and/or fluorescent detection (preferably for colorimetric or photometric detection, e.g., Horseradish-Peroxidase (HRP) or alkaline phosphate (AP)) allow expression of such EVs and proper subsequent assays, e.g. even after tissue fixation. That is, they allow identification of transfected cells without obscuring the intracellular ultrastructure or organelles and in particular allows identification of synaptic sites using electron microscopy. Turning a heterologous marker like HRP into a functional, EV-tethered enzyme—as has been done in context with the present invention—was surprising because HRP needs hem as cofactor in the active center during redox reactions. This was known for the soluble HRP enzyme but the method described and provided in context with the present invention works even for the EV-targeted, membranous HRP. That is, two totally independent read-out methods for EV quantitation (substrate turnover and antibody-mediated direct detection; cf. (c1) and (c2) of the method described and provided herein) based on the identical protein domain, which is freely accessible (preferably) on the surface or the inner side of the control EVs.

Also, unlike as for chemiluminescence based assays or fluorescent based assays with intraluminal marker protein fusions (cf., e.g., WO2019091964), for the method of the present invention there is no lysis of the EVs needed if said heterologous marker suitable for colorimetric, photometric, and/or fluorescent detection (preferably colorimetric or photometric detection, e.g., HRP) is located on the outer side of the control EV.

Without being bound by theory, lysis of the EVs is deemed not to be required, if said heterologous marker suitable for colorimetric, photometric, and/or fluorescent detection (preferably colorimetric or photometric detection, e.g., HRP) is located on the inner side of the control EV. This is because; suitable substrates for colorimetric, photometric, and/or fluorescent detection (preferably colorimetric or photometric detection, e.g., HRP) may be available which are able to pass or cross the membrane(s) of the EVs.

Furthermore, as has surprisingly turned out in context with the present invention, unlike as for fluorescent based assays with intraluminal marker protein fusions, using control EVs as described herein (e.g., EVs with a heterologous marker suitable for colorimetric photometric detection, e.g. HRP) are inert regarding the formation of a so-called protein corona (a layer of serum or plasma proteins that is believed to affect the accessibility of antibodies to EV surface proteins). It has been found in context of the present invention that control EVs as described herein (e.g., EVs with a heterologous marker suitable for colorimetric or photometric detection, e.g. HRP) are completely insensitive to this phenomenon and the enzymatic activity is not compromised even in undiluted serum samples spiked with control EVs as described herein (e.g., EVs with a heterologous marker suitable for colorimetric photometric detection, e.g. HRP).

Furthermore, as has surprisingly turned out in context with the present invention, unlike as for chemiluminescence or bioluminescence based systems (cf., e.g., US20130078658) which produce light only during the chemical redox reaction, control EVs as described herein (e.g., EVs with a heterologous marker suitable for colorimetric or photometric detection, e.g. HRP) can use substrates that can be detected by colorimetric/photometric or fluorometric principles which accumulate over time and which can be measured minutes or even hours after addition of the substrate of choice such that the accumulation of the processed substrate increases the sensitivity of detection massively. In contrast, light emission by fluorescence and chemiluminescence is transient and is not linear over time because fluorescence suffers from bleaching and chemiluminescence shows a very sharp initial peak followed by a rather rapid decline which needs a time-resolved measurement to generate real numbers in relation to a reference, making it difficult to standardize/normalize quantitative results.

In sum, EVs comprising a heterologous marker wherein said marker is located (i) on the outer side or (ii) on the inner side of the control extracellular vesicle and which is suitable for colorimetric, photometric, and/or fluorescent detection provide for advantageous properties as described above, which could not have been expected.

In fact, the prior art put emphasis on EVs labelled with a marker providing for bioluminescence or chemiluminescence in order to monitor EVs (as a control) “in situ” or “live”. The prior art achieved this, e.g. by labelling EVs with fluorescent proteins or with luciferase. Yet, although both chemiluminescence and bioluminescence allow monitoring the quality of assays comprising EVs, they both have the disadvantage that they are not readily suitable for a quantitative analysis of assays comprising EVs. Indeed, (bio/chemi) luminescence produces light that radiates non-directionally only during the bio/chemical redox reaction. As a consequence, no product accumulates over time but great care has to be taken to choose the appropriate catalyst/substrate (plus enhancers in certain cases) and to ensure the correct timing after adding it. This is due to the fast reaction rate and its spike-like kinetics followed by an exponential decay of signal intensity. Integrating the signal (e.g., light) over time is essential to “catch” the light in the correct time window. Moreover and under certain conditions (e.g., the need to be highly sensitive due to a low signal), the catalyst/substrate needs to be “injected” into the sample to record the emitted light during the optimal time frame after the start (i.e. injection of the substrate) of the redox reaction and to guarantee reproducible reaction conditions.

In view of the above, in context of the present invention, detection of EVs by a principle making use of chemiluminescence or bioluminescence or biofluoresecnce or fluorescence, e.g. through light-emitting proteins as such is preferably excluded. The term “light-emitting protein” as used before refers to a protein that emits light by a change in physical conditions or by a chemical process. Non-limiting examples of such proteins are fluorescent proteins such as green fluorescent protein, enhanced green fluorescent protein, yellow fluorescent protein and red fluorescent protein, luminescent proteins, photoproteins or luciferase.

In contrast, substrates that can be detected by colorimetric/photometric or fluorometric principles accumulate over time and can be measured minutes/hours after addition of the substrate of choice such that the accumulation of the processed substrate increases the sensitivity of detection massively. This is the case, e.g. in particular with oxidases or peroxidases, such as with HRP and other peroxidases, but also with alkaline phosphatase, for example.

Accordingly, in one embodiment of the present invention, the amount of control extracellular vesicles detected in step (c) after the isolation step is quantitatively determined in step (d). In another embodiment of the present invention, the amount of control extracellular vesicles detected in step (c) after the isolation step is determined in step (d) at a predefined point of time after the isolation step. As described above, this is possible in accordance with the present invention, since the processed substrates of peroxidases accumulate over time and can be measured virtually at any time since accumulation of processed substrates is linear within a wide range of conditions. In principle and in accordance with the present invention, the processed substrate can be measured at a discrete time point after having added the substrate. There is no lag time that needs to be taken into consideration and the discrete time point can be minutes (preferably 5 to 4 h, more preferably 5 to 60 min, more preferably 10 to 30 min). Also, in accordance with the present invention, accumulation of substrate may increase sensitivity with time. Accordingly, in another embodiment of the present invention, the amount of control extracellular vesicles detected in step (c) after the isolation step is determined in step (d) without lag time.

Accordingly, it is preferred that in the methods of the present invention when detecting the amount of control extracellular vesicles among the isolated extracellular vesicles (e.g., step (c) or step (c1) as mentioned herein) a fluorogenic or chromogenic substrate is added. Thus, in a preferred embodiment, the methods of the present invention further comprise in step (c) or step (c1) adding a fluorogenic or (preferably) chromogenic substrate. It is preferred in context of the present invention that chemiluminescent or bioluminescent substrates are excluded.

In one embodiment of the present invention, said substrate processed by the heterologous marker accumulates over time. In accordance with the present invention, accumulation of the processed substrate is linear with time. In general, processed substrate can be measured at a discrete time point after the addition of the substrate. In a further embodiment of the present invention, the amount of EV detected in step (c) is determined 5 min to 4 hours preferably 5 to 60 min, more preferably 10 to 30 min) after the isolation step (b).

For the control extracellular vesicles as described herein and as to be employed in the methods or contained in the kits described and provided herein, said heterologous marker wherein said marker can be located (i) on the outer side or (ii) on the inner side of the control extracellular vesicle. Preferably, said heterologous marker is located on the outer side of the control EV, e.g., if lysis of the EVs should be avoided in the detection assay and/or substrates are used which cannot pass through the EV membrane.

However, in one embodiment of the invention, said heterologous marker is located on the outer side of the control EV. This may for example be possible or even desirable where substrates are used that can pass the EV membrane, and/or where large amounts of proteins are present in the EVs. That is, without being bound by theory, it may be advantageous to have the heterologous marker located on the inner side of the control extracellular vesicle, if a biofluid sample to which control extracellular vesicles are mixed contains a high amount of proteins.

In a preferred embodiment of the present invention, step (c) of detecting the amount of control extracellular vesicles among the isolated extracellular vesicles is done using a colorimetric photometric, or fluorescent detection assay (preferably a colorimetric or photometric detection assay) as defined in step (c1) of the method described and provided herein. However, to detect the heterologous marker protein and thus the amount of control EVs among the isolated EVs, it is also possible in context with the present invention to use an immuno-based assay (e.g., ELISA, EIA, Western Blot, ELISpot, and other suitable immune-based assays as known in the art) employing a binding agent which (specifically) binds to said heterologous marker, wherein said marker is located on the outer side. The control EVs as described herein (e.g., EVs with a heterologous marker suitable for colorimetric photometric detection, e.g. HRP) are fully compatible with immunological, antibody-based standard assays such as sandwich ELISAs and other immune-based assays as known in the art and as described herein, and said control EVs as described herein may also carry conventional surface proteins characteristic for EVs such as CD63, CD9, CD81, and others and which are detectable by said immuno-based assays (e.g., ELISA, EIA, Western Blot, ELISpot, and other suitable immune-based assays as known in the art).

The term “specifically binding” (equally used herein with “specifically directed to”) means in accordance with this invention that the recognition molecule is capable of specifically interacting with and/or binding to at least two, preferably at least three, more preferably at least four amino acids of an epitope as defined herein. Such binding may be exemplified by the specificity of a “lock-and-key-principle”. Thus, the term “specifically” in this context means that the recognition molecule binds to a given target epitope but does not essentially bind to another protein. The term “another protein” includes any protein including proteins closely related to or being homologous to the epitope against which the recognition molecule is directed to. However, the term “another protein” does not include that the recognition molecule cross-reacts with the epitope from a species different from that against which the recognition molecule was generated. The term “cross-species recognition” or “interspecies specificity” as used herein means binding of a binding domain described herein to the same target molecule in humans and non-human species. Thus, “cross-species specificity” or “interspecies specificity” is to be understood as an interspecies reactivity to the same epitope expressed in different species, but not to another molecule other than X.

A “binding agent” as used herein can be any molecule which is capable of (specifically) binding to a polypeptide. In one embodiment of the present invention, a binding agent can be an antibody or a fragment thereof (e.g., Fab, Fab′, Fv, scFv, F(ab′)2). An “antibody” as used herein is a protein comprising one or more polypeptides (comprising one or more binding domains, preferably antigen binding domains) substantially or partially encoded by immunoglobulin genes or fragments of immunoglobulin genes. The term “immunoglobulin” (lg) is used interchangeably with “antibody” herein. The recognized immunoglobulin genes include the kappa, lambda, alpha, gamma, delta, epsilon and mu constant region genes, as well as myriad immunoglobulin variable region genes.

In particular, an “antibody” when used herein, is typically tetrameric glycosylated proteins composed of two light (L) chains of approximately 25 kDa each and two heavy (H) chains of approximately 50 kDa each. Two types of light chain, termed lambda and kappa, may be found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. The term “antibody” as used herein includes antibodies that compete for binding to the same epitope as the epitope bound by the antibodies of the present invention, preferably obtainable by the methods for the generation of an antibody as described herein elsewhere. The term “antibody” also includes but is not limited to polyclonal, monoclonal, monospecific, polyspecific such as bispecific, non-specific, humanized, human, single-chain, chimeric, synthetic, recombinant, hybrid, mutated, grafted, and in vitro generated antibodies, with a polyclonal antibody being preferred. Said term also includes domain antibodies (dAbs) and nanobodies. Accordingly, the term “antibody” also relates to a purified serum, i.e. a purified polyclonal serum. Accordingly, said term preferably relates to a serum, more preferably a polyclonal serum and most preferably to a purified (polyclonal) serum. The antibody/serum is obtainable, and preferably obtained, for example, by the method or use described herein. “Polyclonal antibodies” or “polyclonal antisera” refer to immune serum containing a mixture of antibodies specific for one (monovalent or specific antisera) or more (polyvalent antisera) antigens which may be prepared from the blood of animals immunized with the antigen or antigens. Furthermore, the term “antibody” as employed in the invention also relates to derivatives or variants of the antibodies described herein which display the same specificity as the described antibodies. Examples of “antibody variants” include humanized variants of non-human antibodies, “affinity matured” antibodies (see, e.g., Hawkins et al., J Mol Biol (1992), 254, 889-896; and Lowman et al., Biochemistry (1991), 30:10832-10837) and antibody mutants with altered effector function(s) (see, e.g., U.S. Pat. No. 5,648,260). The term “monoclonal antibody” as used herein refers to an antibody obtained from a population of substantially homogeneous antibodies, i.e. the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature (1975), 256:495, or may be made by recombinant DNA methods (see, e.g., U.S. Pat. No. 4,816,567). The “monoclonal antibodies” may also be isolated from phage antibody libraries using the techniques described in Clackson et al., Nature (1991), 352:624-628; and Marks et al., J Mol Biol (1991), 222:581-597, for example.

In one embodiment of the method described and provided in context with the present invention, the heterologous marker is a protein. In specific embodiments of the present invention, the heterologous marker is selected from the group consisting of peroxidase, alkaline phosphatase beta-galactosidase, glycosidase, caspase, granzyme B, HIV protease, elastase, collagenase, chloramphenicol acetyltransferase, and lipase. In a more specific embodiments of the present invention, the heterologous marker is a peroxidase, preferably Horseradish Peroxidase (HRP) or any isoenzyme thereof as known in the art (e.g., Shannon et al., J Biol Chem (1966), 241:2166-2172).

Preferred substrates for alkaline phosphatase include fluorescein disphosphate, dimethylacradinone phosphate, methylumbelliferyl phosphate, ELF 97 phosphate, NBT/BCIP, and Amplex red.

Preferred substrates for beta-galactosidase include difluorescein digalactoside, resorufin galactoside, DDAO galactoside, methylumbelliferyl galactoside, 5-chloromethylfluorescein di-β-D-galactopyranoside (CMFDG), and 5-(Pentafluorobenzoylamino)fluorescein di-β-D-galactopyranoside (PFB-FDG).

HRP provides for a superior readout, as it is a highly active enzyme (turnover of substrate leads to accumulation of the product) and its presence can be easily monitored. Accordingly, in context of the methods of the present invention, at each step after spiking a sample, HRP-EVs can be traced and quantitated by simply adding substrate followed by a short incubation time (e.g. minutes) and a photometric/colorimetric readout. Employing the method described in provided in context with the present invention allows a much more sensitive approach, e.g. detecting as few as app. 10⁵ to 10⁶ control EVs among 10¹⁰ to 10¹¹ sample EVs. In contrast, in the prior art (e.g. WO 2019091964), identification is based on the presence of GFP in the EVs and ratio of nanotracking analysis (NTA) versus fluorescent NTA (fNTA) signals needs to be determined using a specialized nanotracking analysis instrument. Also, that technology cannot be adapted to high throughput processes as it is slow, unprecise and needs constant supervision. Besides, using bioluminescence or chemiluminescence needs a very high input of, e.g. EVs with fluorescent proteins, such as gag-eGFP-EVs or EVs labelled with, e.g. luciferase, to score in the fNTA channel (e.g., presumably >10%, i.e., 10¹⁰ gag-eGFP-EVs/mL in a complex sample that contains ≥10¹¹ EVs/mL such as serum).

Using HRP as marker protein as described herein, preferably in combination with HRP-specific antibodies, HRP-EVs can be removed at each step of analysis or prior to further sample processing, if required. In the prior art, e.g. WO 2019091964 gag-eGFP-EVs need to be modified—PEGylated EVs—to carry an external tag that can be used in combination with PEG-specific antibodies for their removal. As the input of gag-eGFP-EVs is high [in some experiments in the order of 10 to 20%] removal is a must for certain readouts.

Moreover, HRP-EVs are fully compatible with immunological, antibody-based standard assays such as sandwich ELISAs, because the HRP-EVs carry also conventional surface proteins characteristic of EV such as CD63, CD9, CD81, etc., Immobilization or capture of our HRP-EVs together with EVs in the sample to be analyzed is possible and compatible with the HRP readout (substrate turnover) in this assay format.

The prior art, e.g. WO 2019091964 is not compatible with sandwich ELISA assay format, immobilization of gag-eGFP-EVs disables the direct readout of particle numbers [ratio of EVs enumerated by NTA versus EVs enumerated by fNTA]. As a substitute, the gag domain can be used, but its detection requires the lysis of gag-eGFP-EVs and the subsequent immobilization of gag to be analyzed in a sandwich ELISA.

In sum, particularly HRP-EVs provide for unexpected superiority versus EVs labelled, e.g. with fluorescent or bioluminescent markers.

The term “polypeptide” is equally used herein with the term “protein”. Proteins (including fragments thereof, preferably biologically active fragments, and peptides, usually having fewer than 30 amino acids) comprise one or more amino acids coupled to each other via a covalent peptide bond (resulting in a chain of amino acids). The term “polypeptide” as used herein describes a group of molecules which typically comprise more than 15 amino acids. Polypeptides may further form multimers such as dimers, trimers and higher oligomers, i.e. consisting of more than one polypeptide molecule.

In one embodiment of the present invention, the control extracellular vesicles further comprise a membrane protein, and said heterologous marker is connected to said membrane protein. Said heterologous marker can be connected to the membrane protein in any way as known in the art. In specific embodiments of the present invention, said heterologous marker can be directly fused to said membrane protein (preferably recombinantly, via genetic fusion and joint translation), covalently linked to said membrane protein, linked via click chemistry, or connected to said membrane protein via a linker. Such linker can be of any kind known in the art, e.g., flexible linkers, rigid linkers or in vivo cleavable linkers (see, e.g., Chen et al., Adv Drug Deliv Rev (2013), 65(10): 1357-1369). Preferably, such linkers as used in context with the present invention are in vivo non-cleavable. Examples of linkers usable in context with the present invention include short peptide linkers (e.g., peptides comprising only 2-6, preferably 2-4 amino acids) or (poly)peptide linkers and chemical linkers, preferably short peptide linkers or (poly)peptide linkers. The linker can also serve as docking-site for other binding agents. In this context, and in accordance with the present invention, a linker could also be, e.g., streptavidin or another polypeptide serving as docking-site for biotin. In a specific embodiment of the present invention, said heterologous marker can be connected to the membrane protein via chemical coupling from the outside as known in the art and described in, e.g., Henna Matero's Master's Thesis “HRP-labeling of bacterial extracellular vesicles for transmission electron microscopy imaging” (2022).

In context with the present invention, where the control extracellular vesicles further comprise a membrane protein, said membrane protein may direct its own release through vesicles as a membrane-bound protein (self-assembling; cf. US20130078658). For example, said heterologous marker protein can penetrate a lipid bilayer. In one embodiment of the present invention, the membrane protein is a cellular membrane protein, for example, an exosomal membrane protein. The membrane protein can be a portion or fragment of a full-length membrane protein sufficient to introduce the fusion protein into the exosomes, particularly the lipid membrane of the exosome. For example, the membrane protein can comprise an N-terminus or C-terminus region of the membrane protein (e.g., about 5 or more, about 10 or more, about 15 or more, about 20 or more, or about 25 or more contiguous amino acids from the C-terminus or N-terminus of a full-length membrane protein). Generally, in accordance with the present invention, origins of membrane proteins can be, e.g., metazoan or protozoan cells, alternatively, enveloped viruses or bacteriophages.

In one embodiment of the present invention, said membrane protein is selected from the group consisting of transmembrane proteins, Gi-coupled proteins, membrane-anchored receptors, tetraspanins, type I or II or III transmembrane proteins, and single or multipass transmembrane proteins. In another embodiment of the present invention, said membrane protein is selected from the group consisting of Gag (HIV) and other retroviral specific antigen (variations), M1 (Influenza), Arrdc1 (non-viral; human), VP40 (Ebola), Arc (non-viral; human), M protein (VSV), EpCAM, CD63, CD37, CD53, CD81, CD82, CD54 (ICAM1), CD9, CD151, TSPAN-8 (tetraspanins), Lactadherin (MFGE8), Hsc70, MHC I, Tsg101, calnexin, gp96, L1, integrins such as alpha-3, -5, -V, -6 and beta-1 and -3, and type I or II membrane proteins. In a specific embodiment of the present invention, said membrane protein is selected from the group consisting of CD63 as it is enriched in EVs and Lactadherin (MFGE8).

In a specific embodiment of the present invention, said heterologous marker is HRP, said detection step (c) is carried out in the presence of a substrate of HRP. In context with the methods and the kit described and provided herein, said substrate is preferably a fluorogenic substrate or chromogenic substrate. In a specific embodiment of the present invention, said substrate can be selected from the group consisting of 2,2′-Azino-bis(3-ethylbenzthiazoline-6-sulfonis acid (ABTS), 3-amino-9-ethylcarbazole (AEC), 4-chloro-1-naphthol (4CN), 3,3′-Diaminobenzidine (DAB), 3,3′,5,5′-Tetramethylbenzidine (TMB), o-Phenylenediamine (OPD), Amplex Red (10-acetyl-3,7-dihy-droxyphenoxazine), tyramine, homovanillic acid (HVA), and 4-Hydroxyphenylee acetic acid, preferably from the group consisting of 2,2′-Azino-bis(3-ethylbenzthiazoline-6-sulfonis acid (ABTS), 3-amino-9-ethylcarbazole (AEC), 4-chloro-1-naphthol (4CN), 3,3′-Diaminobenzidine (DAB), 3,3′,5,5′-Tetramethylbenzidine (TMB), 0-Phenylenediamine (OPD). As mentioned above, using substrates for colorimetric or photometric assays can be advantageous in context of the present invention as such substrates, i.e., their processed, enzymatically converted derivatives, accumulate with time while the enzyme still works on processing remaining substrate molecules. Enzymatically converted substrate derivatives can be detected by colorimetric/photometric principles minutes or even hours after addition of the substrate of choice such that the accumulation of the processed substrate increases the sensitivity of detection.

In a further embodiment of the present invention, said control extracellular vesicles further comprise
(iii) streptavidin or biotin (preferably streptavidin as biotin can be present in small amounts in biofluids) located on the outer side of the control extracellular vesicle.

In accordance with the present invention and as described herein, the control extracellular vesicles can comprise streptavidin or biotin (preferably streptavidin) located on the outer side of the control extracellular vesicle either apart from the heterologous marker, or connected to the heterologous marker as described herein. The presence of streptavidin or biotin (preferably streptavidin) can serve as docking-site for biotin or streptavidin (preferably biotin), respectively, thus making the control extracellular vesicle extremely versatile as only one type of streptavidin- or biotin-(preferably streptavidin-) comprising type of extracellular vesicle needs to be prepared which can then be coupled in a second step with almost covalent bond strength with, e.g. an streptavidin-coupled enzyme of choice such as HRP or another peroxidase. Another advantage of this approach is that it allows enzyme coupling after sample processing and isolation of EVs even in complex biomedical fluids.

Generally, in accordance with the present invention, said biofluid sample comprising sample extracellular vesicles can be of any suitable origin. In one embodiment of the present invention, the biofluid sample is selected from the group consisting of plasma sample, serum sample, urine sample, saliva sample, tear drop sample, cerebrospinal fluid sample, spinal fluid sample, peritoneal sample, ascites sample, pleural sample, and joint fluid sample, preferably serum and/or plasma samples. Preferably, the sample is of human origin.

In another embodiment of the present invention, said control extracellular vesicles may further comprise a protein of interest whose presence or overexpression is considered to represent a medical condition. For example, such protein of interest may be a tumor marker protein which is considered to be indicative for the presence of or increased risk for developing a certain tumor. In this context, in a further embodiment of the present invention, the method to determine the recovery rate of sample extracellular vesicles as described and provided herein may further comprise a step of detecting said protein of interest as described herein. The detection of said protein of interest may be conducted in any suitable way known in the art, including, e.g., employing an immune-based assay such as ELISA, EIA, Western Blot, ELISpot, and other.

The present invention further relates to a kit comprising a control extracellular vesicle as described herein above and below. In context with the present invention, such a kit may further comprise a fluorogenic substrate or chromogenic substrate. In a specific embodiment of the present invention, said substrate can be selected from the group consisting of 2,2′-Azino-bis(3-ethylbenzthiazoline-6-sulfonis acid (ABTS), 3-amino-9-ethylcarbazole (AEC), 4-chloro-1-naphthol (4CN), 3,3′-Diaminobenzidine (DAB), 3,3′,5,5′-Tetramethylbenzidine (TMB), o-Phenylenediamine (OPD), Amplex Red (10-acetyl-3,7-dihy-droxyphenoxazine), tyramine, homovanillic acid (HVA), and 4-Hydroxyphenylee acetic acid, preferably from the group consisting of 2,2′-Azino-bis(3-ethylbenzthiazoline-6-sulfonis acid (ABTS), 3-amino-9-ethylcarbazole (AEC), 4-chloro-1-naphthol (4CN), 3,3′-Diaminobenzidine (DAB), 3,3′,5,5′-Tetramethylbenzidine (TMB), o-Phenylenediamine (OPD). Also, in a further embodiment in this context and in accordance with the present invention, the kit may further comprise a binding agent as described herein which (specifically) binds to said heterologous marker comprised by the control extracellular vesicle. In one embodiment of the present invention, the binding agent itself may further comprise a heterologous marker (e.g., HRP) as described herein.

The present invention further relates to a method for preparing control extracellular vesicles as described herein, comprising the steps
(i) introducing an expression system encoding said heterologous marker into a host cell,
(ii) culturing said cells in a medium, and
(iii) recovering released control extracellular vesicles from said medium.

According to the present invention, introduction of the expression system encoding said heterologous marker into a host cell in step (i) can be done in any suitable manner known in the art. Examples for introducing said system into a host cell include, e.g., (stable) transduction, transfection, or transformation of the expression system into the host cell. As used herein, an “expression system” is preferably a nucleic acid molecule.

Generally, as used herein, the terms “nucleic acid molecule”, “nucleic acid” and “polynucleotide” are to be construed synonymously. Generally, nucleic acid molecules may comprise inter alia DNA molecules, RNA molecules, oligonucleotide thiophosphates, substituted ribo-oligonucleotides or PNA molecules. Furthermore, the term “nucleic acid molecule” may refer to DNA or RNA or hybrids thereof or any modification thereof that is known in the art incl. LNAs (locked nucleic acids) (see, e.g., U.S. Pat. Nos. 5,525,711, 4,711,955, 5,792,608 or EP 302175 for examples of modifications). The polynucleotide sequence may be single- or double-stranded, linear or circular, natural or synthetic, and without any size limitation. For instance, the polynucleotide sequence may be genomic DNA, cDNA, mitochondrial DNA, mRNA, antisense RNA, ribozymal RNA or a DNA encoding such RNAs or chimeroplasts (Gamper, Nucleic Acids Research, 2000, 28, 4332-4339). Said polynucleotide sequence may be in the form of a vector, plasmid or of viral DNA or RNA. Also described herein are nucleic acid molecules which are complementary to the nucleic acid molecules described above and nucleic acid molecules which are able to hybridize to nucleic acid molecules described herein. A nucleic acid molecule described herein may also be a fragment of the nucleic acid molecules in context of the present invention. Particularly, such a fragment is a functional fragment. Examples for such functional fragments are nucleic acid molecules which can serve as primers.

In accordance with the present invention, the heterologous marker can thus be provided by direct transfer into producer cells using cell-transfection procedures known to a person skilled in the art. For heterologous markers consisting of RNA, this molecule can be supplied either in its native form (i.e. unprotected) or in a protected form. Protected RNA relates to modifications in the RNA molecule that enhance the stability of the molecule by reducing RNase activity (see Fisher et al., Nucleic Acids Res (1993), 21:3857-3865). Examples of modifications are phosphorothioate bonds, 2′-0-methyl bases and 2′fluoro bases. Alternatively, the heterologous RNA molecule can be transcribed from an expression cassette, either transfected in cells as a double stranded DNA fragment, as part of a plasmid, or after integration of the expression cassette in the genome. In its simplest form, an expression cassette consists of a promoter, a sequence that is transcribed and a sequence that terminates transcription. Additional elements can be added to improve the transcription process: 1) The transcribed DNA fragment can be fused directly to the DNA that encodes the self-assembling protein; 2) The transcribed DNA encodes a heterologous marker protein as described herein that is translated as a fusion protein to the self-assembling protein. The heterologous RNA marker molecule can be recruited to the vesicles by a-specific binding to the self-assembling protein (see Comas-Garcia et al., eLife (2017), 6: 1-27) or by specific recruitment to the self-assembling protein via the retroviral packaging signal psi (see Comas-Garcia et al., loc cit). Alternatively, the heterologous marker is recruited by a nucleotide-binding polypeptide that recognizes a specific nucleotide target sequence. To this end, the nucleotide-binding polypeptide can be fused directly to the self-assembling protein. Nonlimiting examples of RNA-binding polypeptides are Lambda N, RNA-binding domain of MS2, NS3 and Cas9. The nucleotide target sequence can be used directly as heterologous marker, or can be fused to the heterologous marker sequence. Non-limiting examples of nucleotide target sequences are Nutl, MS2, NS3 aptamers and gRNAs.

In one embodiment of the present invention, suitable host cells into which said expression system is introduced include HEK293 and HEK293T, wherein HEK293 is preferred for stable expression and HEK293T is preferred for transient expression (particularly using plasmids carrying SV40 origins such as, e.g., pcDNA3.1(+)). In accordance with the present invention, cultivation of the cells into which said expression system has been introduced according to step (ii) can be done in any medium suitable for the respective host cells. Preferably, both introduction of said expression system into the cells according to step (i) and cultivation if said host cells according to step (ii) is done in a manner to allow constant release of control extracellular vesicles into the medium. In a preferred embodiment of the present invention, such medium in which the cells are cultivated according to step (ii) of the method provided herein is free of fetal calf serum (FCS). Such FCS fere medium has the benefit that it is free of markerless (bovine) exogenous EVs.

In a further embodiment of the present invention, in step (i) of the method for preparing control extracellular vesicles as described herein an expression system encoding a membrane protein as defined herein is introduced into said host cell. In this embodiment, said expression system encoding a membrane protein may either be the same as that encoding said heterologous marker, or be another expression system, i.e. the molecules (nucleic acid molecules) encoding said heterologous marker and said membrane protein may be the same molecule or be two different molecules.

The present invention further relates to an alternative method for preparing control extracellular vesicles as described herein, comprising the steps
(i)′ adding a heterologous marker as defined herein to extracellular vesicles to chemically conjugate said heterologous marker to said extracellular vesicles in a suitable medium, and
(ii)′ recovering control extracellular vesicles with conjugated heterologous markers from said medium.

In this context and in accordance with the present invention, said heterologous marker can be chemically conjugated by any method known in the art, e.g., as described in Henna Matero's Master's Thesis “HRP-labeling of bacterial extracellular vesicles for transmission electron microscopy imaging” (2022). In a further embodiment of the alternative method for preparing control extracellular vesicles as described herein, said control extracellular vesicles may further comprise a membrane protein as defined herein. For example, the cells from which the EVs are derived from may naturally encode such proteins. In another embodiment of the present invention, an expression system encoding such membrane protein has been introduced (e.g., via (stable) transduction, transfection or transformation) to the cells releasing said EVs.

The present invention further relates to a method to calibrate a device for sample extracellular vesicles analysis, the method comprising:
(a) mixing a biofluid sample as described herein comprising sample extracellular vesicles with a known amount of control extracellular vesicles, wherein the control extracellular vesicles comprise:
   a heterologous marker wherein said marker is located (i) on the outer side or (ii) on the inner side (preferably on the outer side) of the control extracellular vesicle and which is suitable for photometric, colorimetric, and/or fluorescent detection (preferably colorimetric or photometric detection), to obtain a mixture,
(b) introducing said mixture into said device,
(c) analyzing a property of said control extracellular vesicles in said mixture,
(d) optionally analyzing said property again with one or more new combinations of device settings,
(e) deriving from said analysis of said control extracellular vesicles a combination of device settings which gives the exact property of said control extracellular vesicles used to obtain said mixture, and
(f) using said combination of device settings to calibrate the device against future subsequent analysis of samples containing extracellular vesicles with unknown properties, to determine the exact properties thereof.

The term ‘to calibrate’ as used herein means to define the appropriate settings of a device to analyze sample EV.

The term ‘a property’ as used herein means the concentration and/or a specific characteristic like the size, the morphology, the presence of EV markers, the zeta potential etc., of EV.

Preferably, the method to calibrate a device for sample extracellular vesicles analysis further comprises in step (c) adding a fluorogenic or chromogenic substrate.

The present invention further relates to a method to evaluate the isolation of extracellular vesicles, the method comprising:
(a) mixing a biofluid sample as described herein comprising sample extracellular vesicles with a known amount of control extracellular vesicles, wherein the control extracellular vesicles comprise:
   a heterologous marker wherein said marker is located (i) on the outer side or (ii) on the inner side (preferably on the outer side) of the control extracellular vesicle and which is suitable for photometric, colorimetric, and/or fluorescent detection (preferably colorimetric or photometric detection), to obtain a mixture,
(b) isolating the control extracellular vesicles from said mixture, and
(c) detecting the presence of the control extracellular vesicles isolated from said mixture.

Preferably, the method to evaluate the isolation of extracellular vesicles further comprises in step (c) adding a fluorogenic or chromogenic substrate. It is preferred in context of the present invention that chemiluminescent or bioluminescent substrates are excluded.

The embodiments which characterize the present invention are described herein, shown in the Figures, illustrated in the Examples, and reflected in the claims. It is preferred in context of the present invention that chemiluminescent or bioluminescent substrates are excluded.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% or 2% of a given value or range, and also comprise the respective exact numeric value.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### FIGURES

The Figures show:

FIG. 1 shows the structure of three HRP fusion proteins. The structure of three HRP fusion proteins A, B and C is shown with domains derived from the human CD63 protein and the two Epstein-Barr virus glycoproteins gH (encoded by BXLF2) and gp350 (encoded by BLLF1), respectively. In all constructs, the domains are fused via ‘linker’ sequences composed of the amino acids (aa) serine and glycine. Scale numbers indicate aa residues. The illustrations on the right represent the presumed orientation of the fusion proteins in the EV membrane. TM: transmembrane domain; cyto tail: cytoplasmic domain of gp350; SP: signal peptide; EX: extraluminal; IN: intraluminal; C: C-terminus; N: N-terminus.

FIG. 2 shows the expression of HRP on the surface of a modified HEK293 suspension cell line, HEK293-HRP, as revealed by flow cytometry. A retroviral transduction system was used to achieve stable expression of HRP as a fusion protein according to Construct A in FIG. 1. A stable HEK293 suspension cell line expressing HRP was generated by retroviral transduction followed by column-based magnetic cell sorting for cell membrane anchored HRP. After multiple passages of the sorted cells in serum-free synthetic cell culture medium, HRP surface expression in the retrovirally transduced cells (right panel) was measured by flow cytometry using an anti-HRP Alexa647-coupled antibody and compared to parental HEK293 cells (left panel). SSC-A: area of sideward scatter; numbers within the gates: percentage of HRP-positive cells.

FIG. 3 shows in (A) the concentration and in (B) size distribution of EVs derived from HEK293-HRP suspension cells. (C) shows the HRP enzymatic activity of conditioned cell culture medium obtained from this cell line, and (D) shows an HRP-specific colorimetric ELISA assay. The HEK293-HRP suspension cell line was cultivated for three days in serum-free medium supplemented with 1 μM hemin (bovine origin; H9039 by Sigma-Aldrich). The conditioned medium (CM) containing HRP-positive extracellular vesicles (HRP⁺ EVs) was harvested and centrifuged (10 min at 300 g, 20 min at 5,000 g). Nanoparticle Tracking Analysis was used to determine EV concentration (A) and size distribution (B). HRP activity was measured as a function of volume directly by colorimetric detection (C) or after antibody-mediated immobilization of extracellular vesicles (D).
A: Concentration measurement was performed with five independent EV samples.
B: Size distribution of one representative sample is shown (mean size: 149 nm).
C: TMB substrate was added directly to different volumes of conditioned cell culture medium. The reaction was terminated after 15 min by adding 0.1 M sulfuric acid and absorbance was measured at 450 nm using a plate reader.
D: Wells of an ELISA plate were coated with 50 μL of a solution (5 μg/mL) of an HRP-specific antibody overnight. On the next day, the plate was washed with ELISA wash buffer (PBS/0.05% Tween-20) and blocked with 5% (w/v) milk powder in PBS for two hours at 37 °C. Different indicated aliquots of conditioned medium were diluted in PBS (final volume 50 μL) and added to the wells at 37 °C for two hours. The wells were washed with wash buffer and TMB substrate was added to measure HRP activity. The reaction was stopped after 15 min with sulfuric acid as in A. CM: conditioned medium; OD: optical density.

FIG. 4 shows that the enzymatic activity of HRP in conditioned medium of HEK293-HRP cells physically and biochemically co-purifies and is associated with extracellular vesicles. HRP⁺ EVs contained in 30 mL conditioned medium from HEK293-HRP suspension cells were pelleted by ultracentrifugation (100,000 g, 2 h, 4 °C) and loaded underneath a 4-mL preformed iodixanol density gradient (Optiprep™, Sigma-Aldrich) as described in Gärtner et al., J Extracell Vesicles (2019), 8: 1573051. After ultracentrifugation (100,000 g, 16 h, 4 °C) 16 fractions of 250 μL each were collected from top to bottom.
A: The enzymatic activity in 50 μL of each fraction was measured directly by adding TMB substrate (left panel). In parallel, the concentration of EVs in each fraction was measured by nanoparticle tracking analysis (NTA) (middle panel) and the density of the 16 gradient fractions was determined (right panel).
B: 2 μL of each fraction was spotted on nitrocellulose membranes and HRP and the EV marker proteins CD63 and tsg101 were detected with specific antibodies, as described in Gärtner et al., loc cit.

FIG. 5 shows the separation of HRP⁺ EVs from conditioned medium by size-exclusion chromatography. 500 μL of conditioned medium from HEK293-HRP suspension cells were loaded on a 10 mL Sepharose column (Izon Inc.) and separated by size exclusion chromatography according to the manufacturer's protocol. The enzymatic activity in each fraction was measured by adding TMB substrate.

FIG. 6 shows the calculation of the enzymatic activity by using a HRP standard as an external reference and the HRP activity of purified HRP⁺ EVs as function of absolute EV numbers.
A: A serial dilution of a commercially available, purified HRP enzyme with known HRP activity (263 U/mL, Thermo Fisher) was used to establish a calibration curve that shows the direct correlation between the absorbance of the HRP processed TMB substrate and the enzyme concentration. A trendline was calculated using linear regression function (y=4.14⁻⁵x). HRP was added to 100 μL PBS and HRP activity was measured by adding TMB substrate stopping the reaction after 10 min with sulfuric acid.
B: HRP⁺ EVs contained in 30 mL conditioned medium from HEK293-HRP suspension cells were pelleted by ultracentrifugation (100,000 g, 2 h, 4 °C). The particle concentration was determined by nanoparticle tracking analysis and discrete particle numbers were diluted in 100 μL PBS. HRP activity was determined by adding TMB substrate and stopping the reaction after 10 min with sulfuric acid. OD: optical density; one enzyme unit catalyzes the production of 1 mg of purpurogallin from pyrogallol at 20 °C and pH 6.0 in 20 seconds.

FIG. 7 shows the stability of detecting HRP in human serum spiked with HRP⁺ EVs. 100 μL of a serum pool (mixture of three human sera) and 50 μl of conditioned medium from HRP-expressing HEK293 cells containing approximately 7×10⁷ EVs were mixed and incubated at 4 °C for 0, 24, 48 or 72 hours as indicated. Afterwards, EVs were isolated by size exclusion chromatography (SEC) using a Sepharose 4B-packed spin column. The HRP activities in the isolated EV fractions from the different serum/EV mixtures were measured as a function of volume (1, 2, 5, 10 and 20 μL) in a total volume of 100 μL topped with PBS. The reaction with TMB substrate was stopped after 10 min with sulfuric acid. OD: optical density; SEC: size exclusion chromatography.

FIG. 8 shows an example of how HRP⁺ EVs can be used as an internal reference to back-calculate the original concentration of serum EVs after their preparation with a commonly used EV isolation method. 200 μL of a serum pool (mix of three human sera) and 50 μL of conditioned medium from HRP-expressing HEK293 cells containing approximately 7×10⁷ EVs (corresponding to approx. 50 μU HRP) were mixed and incubated for 10 min. EVs were isolated using the ExoQuick EV isolation kit according to the manufacturer's protocol.
The HRP enzymatic activity was measured in 5, 10 and 20 μl of the serum/HRP⁺ EV mix prior to ExoQuick isolation (‘Input’) and after isolation (‘Output’). Linear regression was used to calculate the slopes (m) of the two graphs. By dividing the slope of the Output (mₒᵤₜₚᵤₜ) by the slope of the Input (mᵢₙₚᵤₜ), the recovery rate of HRP⁺ EVs after their processing via the ExoQuick EV isolation kit was calculated. The example shown resulted in a correction factor of 0.42 (mₒᵤₜₚᵤₜ/m_{Input}=0.42→42%). The EV concentration in the Output fraction of the ExoQuick eluate was measured by NTA. The EV concentration in the original input serum sample was back-calculated using the correction factor (output: 1.6×10¹¹ EVs/mL, input: 1.6×10¹¹ EVs/mL*1/0.42=3.81×10¹¹ EVs/mL)
mₒᵤₜₚᵤₜ: slope of the linear regression line of the EV output sample (here: y=0.0086x); mᵢₙₚᵤₜ: slope of the linear regression line of the EV input sample (here: y=0.0205x); OD: optical density.

The present invention may also be characterized by the following items:
(1) A method to determine the recovery rate of sample extracellular vesicles, the method comprising:
   (a) mixing a biofluid sample comprising sample extracellular vesicles with a known amount of control extracellular vesicles, wherein the control extracellular vesicles comprise
      a heterologous marker wherein said marker is located (i) on the outer side or (ii) on the inner side of the control extracellular vesicle and which is suitable for colorimetric, photometric, and/or fluorescent detection,
      to obtain a mixture,
   (b) isolating the extracellular vesicles from said mixture,
   (c) detecting the amount of control extracellular vesicles among the isolated extracellular vesicles using
      (c1) a colorimetric, photometric, or fluorescent detection assay, or
      (c2) an immuno-based assay employing a binding agent which binds to said heterologous marker, wherein said marker is located on the outer side, and
(d) determining the recovery rate of said sample extracellular vesicles based on the ratio of the amount of control extracellular vesicles detected in step (c) after the isolation step to the known amount of control extracellular vesicles mixed with the biofluid sample prior to the isolation step.
(2) The method according to item 1, wherein said heterologous marker is a protein.
(3) The method according to item 1 or 2, wherein said control extracellular vesicles further comprise a membrane protein, and wherein said heterologous marker is connected to said membrane protein.
(4) The method according to item 3, wherein said heterologous marker is directly fused to said membrane protein, covalently linked to said membrane protein, or connected to said membrane protein via a linker.
(5) The method according to item 3 or 4, wherein said membrane protein directs its own release through vesicles as a membrane-bound protein.
(6) The method according to any one of items 3 to 5, wherein said membrane protein is selected from the group consisting of transmembrane proteins, Gi-coupled proteins, membrane-anchored receptors, tetraspanins, type I or II or III transmembrane proteins, and single or multipass transmembrane proteins.
(7) The method according to any one of the preceding items, wherein said heterologous marker is selected from the group consisting of peroxidase, alkaline phosphatase and beta-galactosidase.
(8) The method according to any one of the preceding items, wherein said heterologous marker is horseradish peroxidase (HRP).
(9) The method according to any one of items 1 to 8, wherein the amount of control extracellular vesicles detected in step (c) after the isolation step is quantitatively determined in step (d).
(10) The method according to any one of items 1 to 8, wherein the amount of control extracellular vesicles detected in step (c) after the isolation step is determined in step (d) at a predefined point of time after the isolation step.
(11) The method according to any one of items 1 to 8, wherein the amount of control extracellular vesicles detected in step (c) after the isolation step is determined in step (d) without lag time.
(12) The method according to any one of the preceding items, further comprising in step (c1) adding a fluorogenic or chromogenic substrate.
(13) The method according to item 12, wherein said substrate processed by the heterologous marker accumulates over time.
(14) The method according to item 12 or 13, wherein accumulation of the processed substrate is linear with time.
(15) The method according to any one of items 12 to 14, wherein the processed substrate can be measured at a discrete time point after the addition of the substrate.
(16) The method according to any one of items 12 to 14, wherein the amount of EV detected in step (c) is determined 5 min to 4 hours after the isolation step (b).
(17) The method according to any one of the preceding items, wherein said heterologous marker is HRP, and wherein said detection step (c) is carried out in the presence of a substrate of HRP, said substrate being a chromogenic substrate.
(18) The method according to any one of the preceding items, wherein said heterologous marker is HRP, and wherein said detection step (c) is carried out in the presence of a substrate of HRP, said substrate being selected from the group consisting of 2,2′-Azino-bis(3-ethylbenzthiazoline-6-sulfonis acid (ABTS), 3-amino-9-ethylcarbazole (AEC), 4-chlor-1-naphthol (CN), 3,3′-Diaminobenzidine (DAB), 3,3′,5,5′-Tetramethylbenzidine (TMB), o-Phenylenediamine (OPD), tyramine, homovanillic acid (HVA), and 4-Hydroxyphenylee acetic acid.
(19) The method according to any one of the preceding items, wherein said control extracellular vesicles further comprise
   (iii) streptavidin or biotin
   located on the outer side of the control extracellular vesicle.
(20) The method according to any one of the preceding items, wherein said biofluid sample is selected from the group consisting of plasma sample, serum sample, urine sample, saliva sample, tear drop sample, cerebrospinal fluid sample, spinal fluid sample, peritoneal sample, ascites sample, pleural sample, and joint fluid sample.
(21) The method according to any one of the preceding items, wherein said control extracellular vesicles further comprise a protein of interest whose presence or overexpression is considered to represent a medical condition.
(22) The method according to item 21, said method further comprising a step of detecting said protein of interest.
(23) A kit comprising a control extracellular vesicle as defined in any one of the preceding items.
(24) The kit according to item 23, further comprising a chromogenic or fluorogenic substrate.
(25) The kit according to item 24, wherein said substrate is selected from the group consisting of 2,2′-Azino-bis(3-ethylbenzthiazoline-6-sulfonis acid (ABTS), 3-amino-9-ethylcarbazole (AEC), 4-chloro-1-naphthol (4CN), 3,3′-Diaminobenzidine (DAB), 3,3′,5,5′-Tetramethylbenzidine (TMB), o-Phenylenediamine (OPD), tyramine, homovanillic acid (HVA), and 4-Hydroxyphenylee acetic acid.
(26) The kit according to any one of items 23 to 25, further comprising a binding agent which binds to said heterologous marker.
(27) A method for preparing control extracellular vesicles according to any one of items 1 to 22, comprising the steps
   (i) introducing an expression system encoding said heterologous marker into a host cell,
   (ii) culturing said cells in a medium, and
   (iii) recovering released control extracellular vesicles from said medium.
(28) The method according to item 27, wherein in step (i) an expression system encoding a membrane protein as defined in any one of items 3 to 6 is introduced into said host cell.
(29) A method for preparing control extracellular vesicles according to any one of items 1 to 22, comprising the steps
   (i)′ adding a heterologous marker as defined in any one of items 1 to 22 to extracellular vesicles to chemically conjugate said heterologous marker to said extracellular vesicles in a suitable medium, and
   (ii)′ recovering control extracellular vesicles with conjugated heterologous markers from said medium.
(30) The method according to item 29, wherein said control extracellular vesicles further comprise a membrane protein as defined in any one of items 3 to 6.
(31) A method to calibrate a device for sample extracellular vesicles analysis, the method comprising:
   (a) mixing a biofluid sample comprising sample extracellular vesicles with a known amount of control extracellular vesicles, wherein the control extracellular vesicles comprise:
      a heterologous marker wherein said marker is located (i) on the outer side or (ii) on the inner side of the control extracellular vesicle and which is suitable for photometric, colorimetric, and/or fluorescent detection, to obtain a mixture,
   (b) introducing said mixture into said device,
   (c) analyzing a property of said control extracellular vesicles in said mixture,
   (d) optionally analyzing said property again with one or more new combinations of device settings,
   (e) deriving from said analysis of said control extracellular vesicles a combination of device settings which gives the exact property of said control extracellular vesicles used to obtain said mixture, and
   (f) using said combination of device settings to calibrate the device against future subsequent analysis of samples containing extracellular vesicles with unknown properties, to determine the exact properties thereof.
(32) A method to evaluate the isolation of extracellular vesicles, the method comprising:
   (a) mixing a biofluid sample comprising sample extracellular vesicles with a known amount of control extracellular vesicles, wherein the control extracellular vesicles comprise:
      a heterologous marker wherein said marker is located (i) on the outer side or (ii) on the inner side of the control extracellular vesicle and which is suitable for photometric, colorimetric, and/or fluorescent detection, to obtain a mixture,
   (b) isolating the control extracellular vesicles from said mixture, and
   (c) detecting the presence of the control extracellular vesicles isolated from said mixture.
(33) The method of item 31 or 32, further comprising in step (c) adding a fluorogenic or chromogenic substrate.

The present invention is further illustrated by the following examples. Yet, the examples and specific embodiments described therein must not be construed as limiting the invention to such specific embodiments.

### EXAMPLES

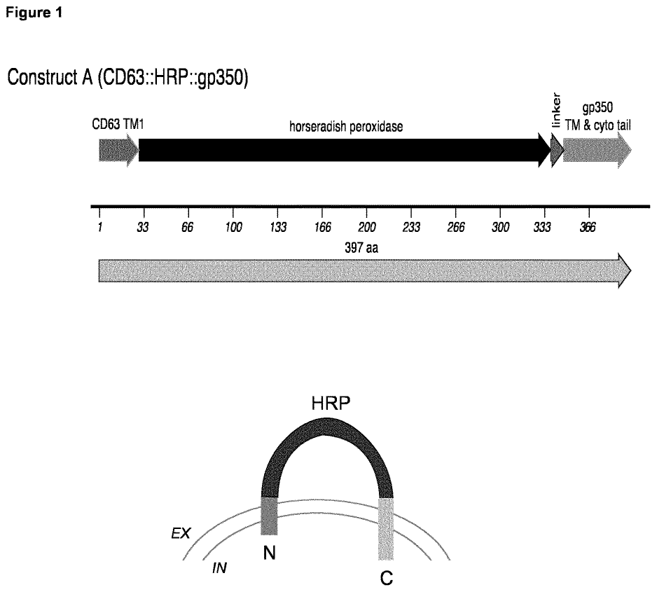

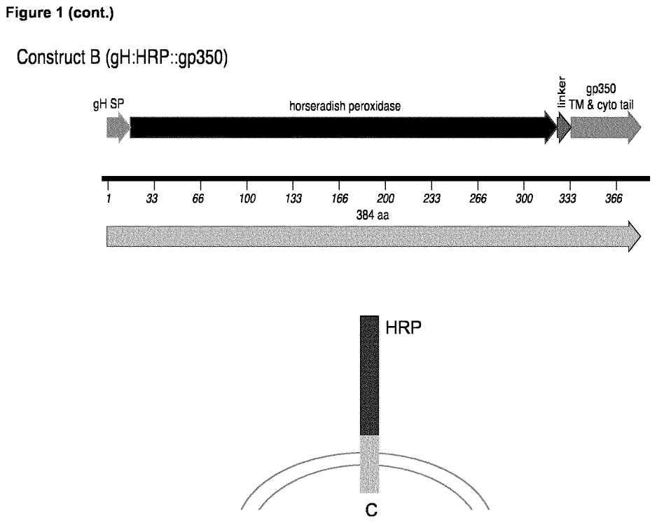

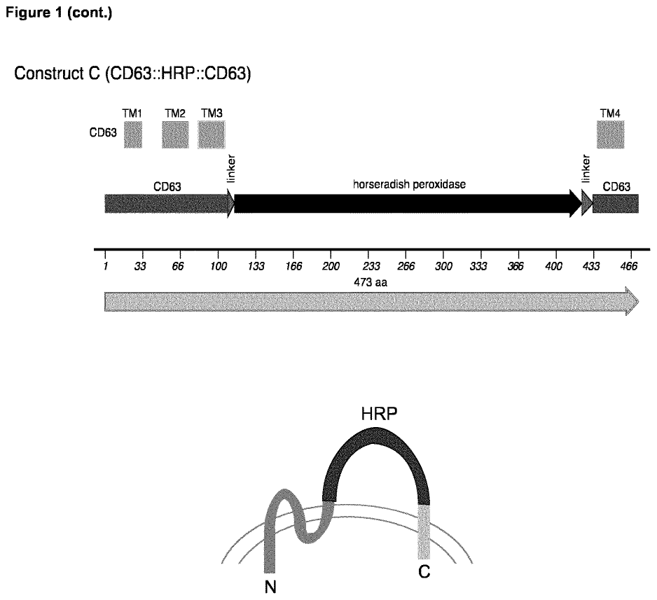

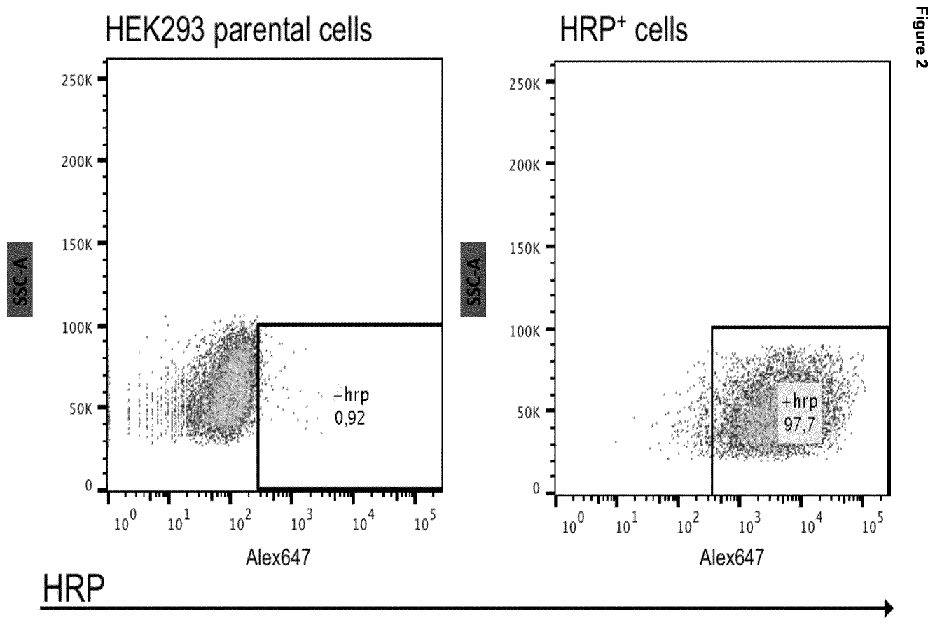

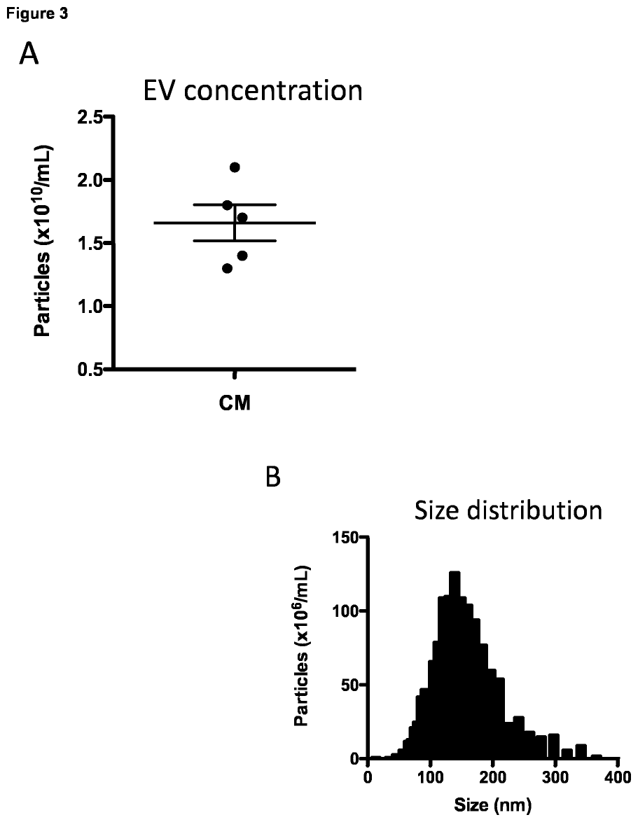

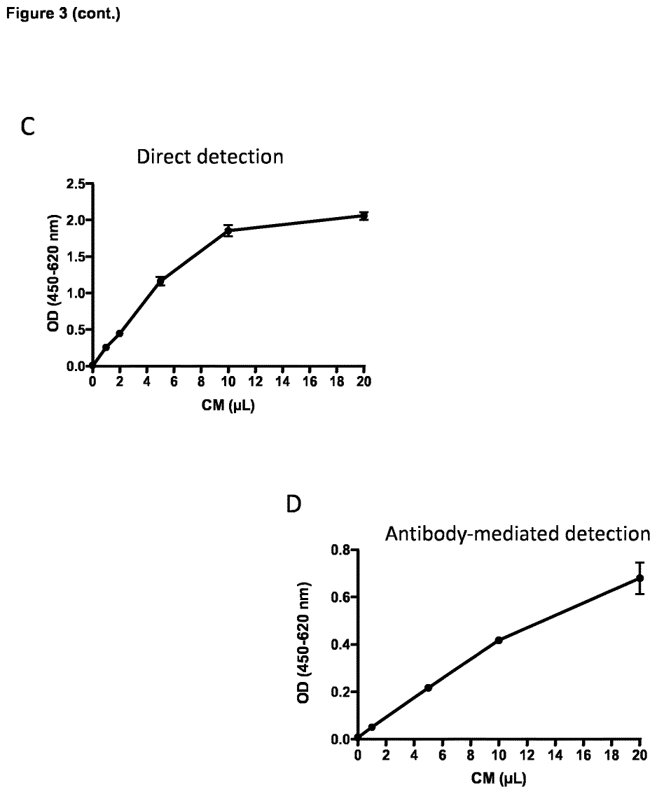

Three fusion proteins with the horseradish peroxidase (HRP) enzyme were prepared to redirect and anchor it into the outer membrane of extracellular vesicles (FIG. 1). In all three constructs, HRP was fused to different protein domains that function as a membrane anchor, hence turning HRP into a functional ectoenzyme on cells and EVs derived from those cells. All sequences were designed by using the MacVector software and synthesized by Genscript using codon optimization for optimal protein expression.

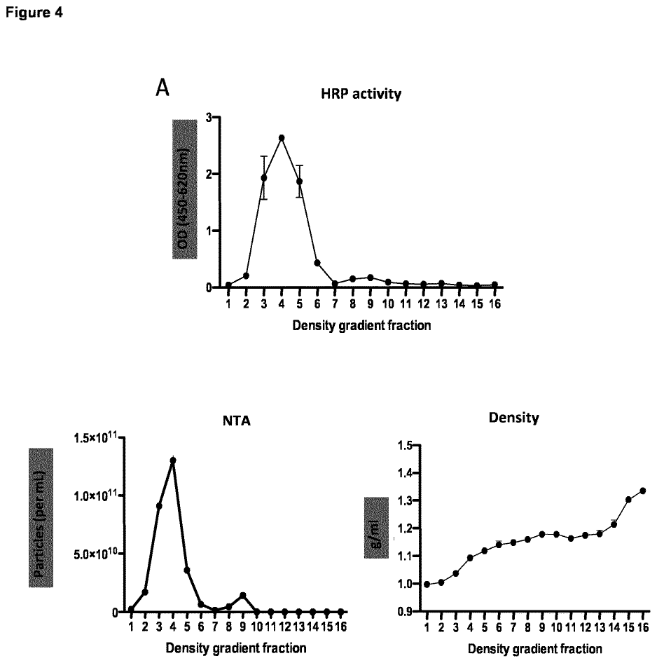

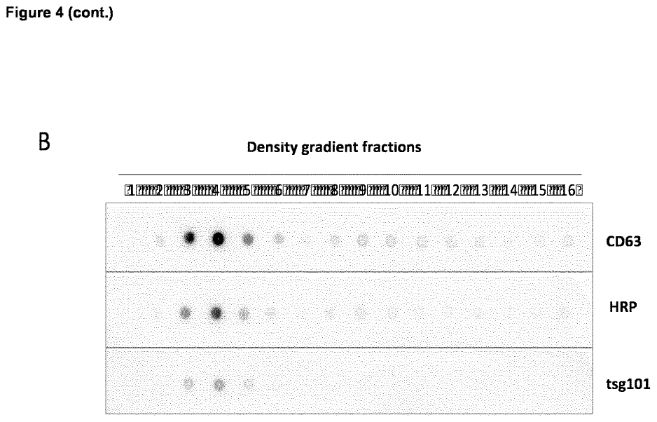

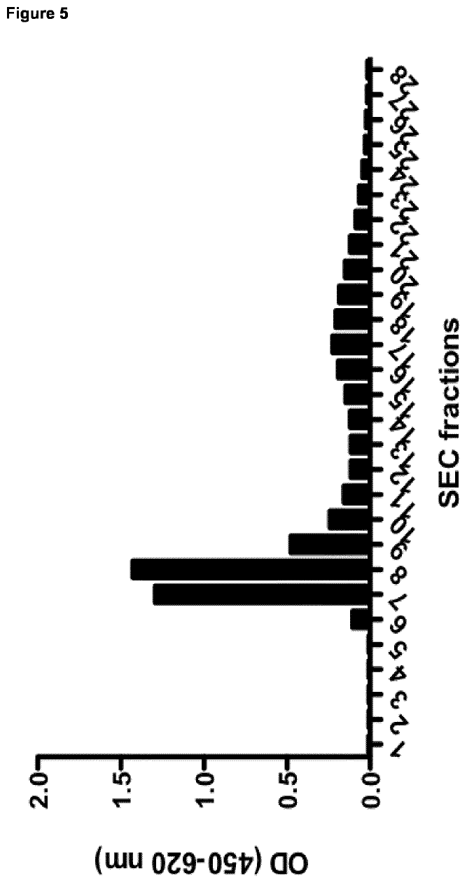

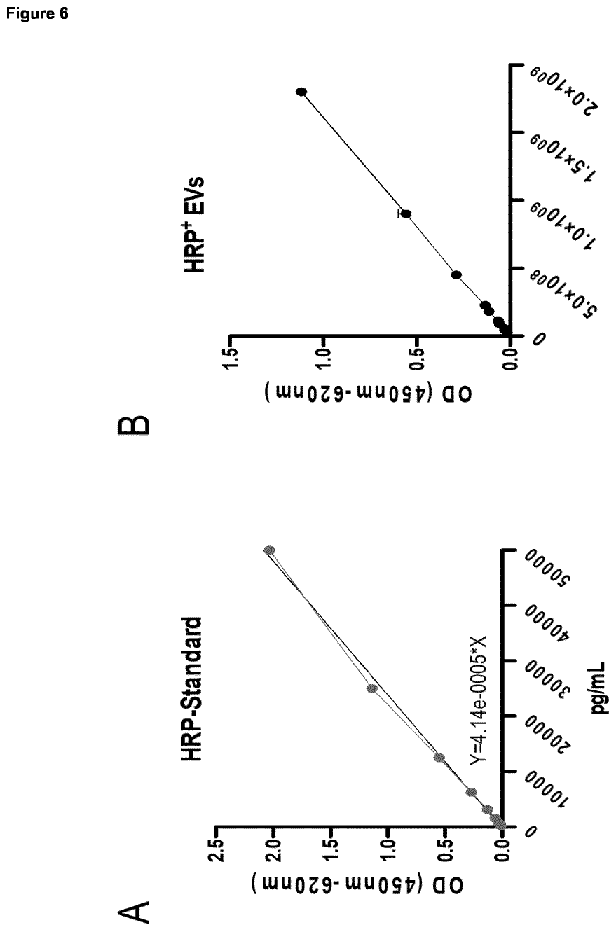

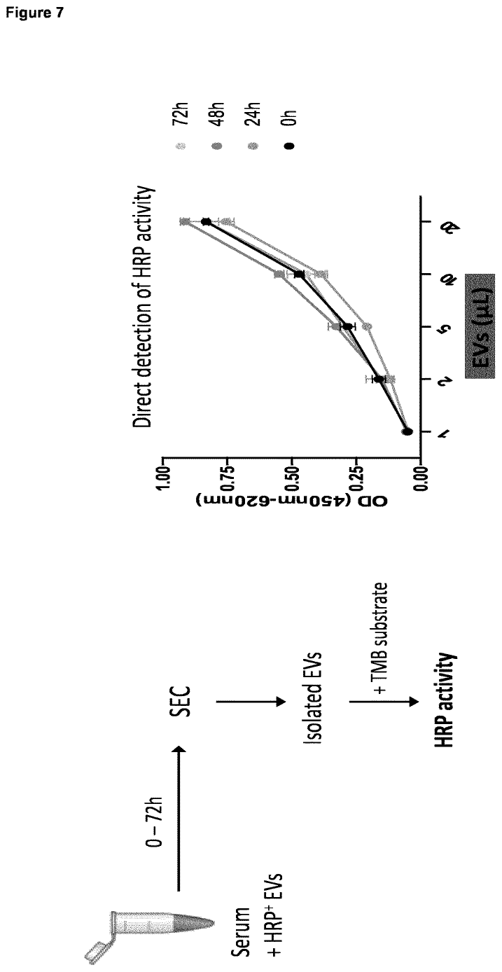

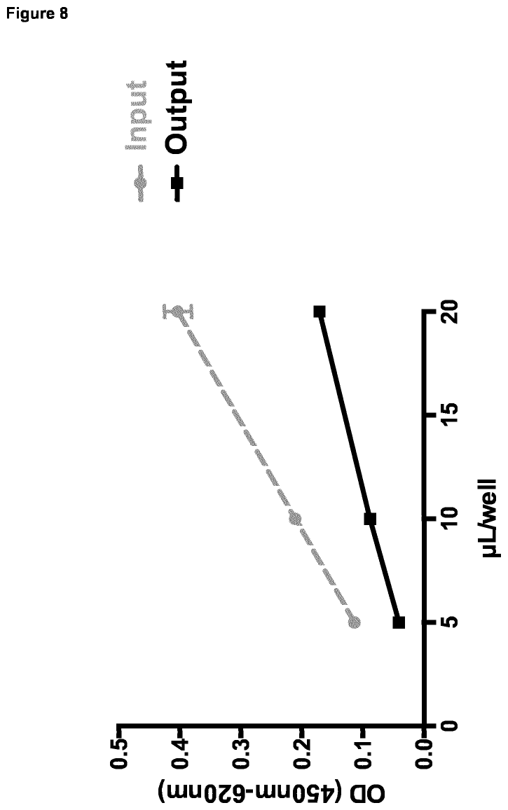

A HEK293 suspension cell line stably expressing HRP was generated by retroviral transduction followed by column-based magnetic cell sorting (MACS MicroBeads Technology, Miltenyi Biotec). High levels of transgene expression on the cell surface were measured by flow cytometry using a HRP-specific antibody (ab10183, Abcam; FIG. 2). To avoid contamination of EVs released from cells with EVs that stem from fetal calf serum (FCS, a constituent of standard cell culture media), the HRP⁺ EV-producing cells were adapted to grow in serum-free, synthetic medium (LV-MAX Production medium, Thermo Fisher). Furthermore, 1 μM of hemin (oxidized form of heme which functions as a cofactor in the HRP enzymatic active center; H9039, Sigma-Aldrich) was added to the medium.

Next, EVs secreted by HEK293-HRP cells into the cell culture medium were characterized (FIG. 3). Cells were seeded at a concentration of 0.5×10⁶ cells/mL and incubated in synthetic medium supplemented with 1 μM hemin for 72 h. The supernatant was harvested, centrifuged and the EV number was enumerated by nanoparticle tracking analysis (ZetaView PMX110, Particle Metrix; FIG. 3A). In parallel the size distribution was determined, showing a mean particle size of 150 nm (FIG. 3B).

The HRP activity in the conditioned cell culture medium (CM) was furthermore quantified by using two independent read-out methods. First, TMB (3,3′,5,5′-tetramethylbenzidine, BD OptEIA, BD Biosciences) was added as HRP substrate to the conditioned medium and substrate turnover was measured by colorimetric detection (plate reader Clariostar, BMG Labtech). Based on absorbance measured in a microplate reader, a linear correlation was shown in a defined concentration range of 6×10⁶-6×10⁷ EVs (correlates to 1-10 μL of CM in FIG. 3C).

In addition, an ELISA plate was coated with an HRP-specific antibody (5 μg/mL, ab10183, Abcam). After washing and blocking the antibody to avoid unspecific binding, different volumes of CM were added to the wells. The immobilized HRP-specific antibodies bind the HRP⁺ EVs selectively, tether them to the solid phase of the ELISA plate. Thereafter, HRP activity was measured by adding substrate to the wells of the ELISA plate. Based on this antibody-mediated detection system, a linearity in the range of 6×10⁶-1.2×10⁸ EVs was shown (FIG. 3D).

To confirm association of HRP in CM with EVs, a density gradient centrifugation was performed as described in the legend of FIG. 4. The fractions of a bottom-up iodixanol gradient (OptiPrep, Sigma-Aldrich) were analyzed by measuring HRP substrate turnover, EV concentration and density (FIG. 4A). Dot blot analysis furthermore confirmed the co-localization of HRP in the typical upper gradient fractions with EVs using the two EV marker proteins CD63 and tsg101 (rat anti-CD63 24F9, Helmholtz Munich, mouse anti-tsg101 4A10, GeneTex; FIG. 4B).

Along the same line, 500 μL of CM were loaded on a Sepharose column (qEV 70 nm, Izon) and separated by size exclusion chromatography (SEC). Subsequent measurement of HRP activity and EV concentration in the collected fractions confirmed the co-purification of EVs and HRP which eluted in the first fractions of the column as expected being excluded from entering the Sepharose beads due to their size (FIG. 5).

The correlation between the number of HRP+ EVs and the corresponding HRP activity was documented by using purified HRP enzyme as a standard (FIG. 6). In the example shown, 1.8×10¹¹ EVs/mL correlated with 0.71 U/ml of HRP.

To investigate the stability of HRP⁺ EVs in complex biological samples, they were incubated in human serum for 0 to 72 h. Then performed SEC was performed and the HRP activity was measured in the EV-containing fractions. No significant influence of the duration of incubation onto HRP activity (FIG. 6) was observed, demonstrating the robustness of this enzymatic detection system, even in biological samples of high protein content.

The yield (recovery) of EVs was evaluated in a commonly used EV purification method (ExoQuick, System Biosciences) that is often used to prepare EVs from e.g. serum samples. A serum sample was spiked with a certain number of HRP⁺ EVs corresponding to 50 μU HRP enzymatic activity. The HRP signal was measured prior to (Input) and after EV purification (Output). The slopes of the linear regression lines were determined and the ratio of the Output HRP signal divided by Input HRP signal resulted in a factor of 0.42 (FIG. 8). Thus, the recovery rate of the ExoQuick was approx. 42%. This correction factor furthermore allows the accurate enumeration of EVs in biological samples as described in the legend of FIG. 8.

### Construction of Synthetic Horseradish Peroxidase (HRP) Genes for the Expression of HRP on the Surface of Extracellular Vesicles (EVs)

The publicly retrievable amino acid sequence of HRP (UniProtKB-P00433 (PER1A_ARMRU); https://www.uniprot.org/uniprotkb/P00433/entry) was used for the assembly of HRP expression plasmids and vectors:

(SEQ ID NO: 1)
MHFSSSSTLFTCITLIPLVCLILHASLSDAQLTPTFYDNSCPNVSNIVRD
TIVNELRSDPRIAASILRLHFHDCFVNGCDASILLDNTTSFRTEKDAFGN
ANSARGFPVIDRMKAAVESACPRTVSCADLLTIAAQQSVTLAGGPSWRVP
LGRRDSLQAFLDLANANLPAPFFTLPQLKDSFRNVGLNRSSDLVALSGGH
TFGKNQCRFIMDRLYNFSNTGLPDPTLNTTYLQTLRGLCPLNGNLSALVD
FDLRTPTIFDNKYYVNLEEQKGLIQSDQELFSSPNATDTIPLVRSFANST
QTFFNAFVEAMDRMGNITPLTGTQGQIRLNCRVVNSNSLLHDMVEVVDFV
SSM

The publicly retrievable aa sequence encompasses a signal peptide leader (aa 1-30), the enzymatic moiety (aa 31-338) and a carboxy-terminal pro-peptide (aa 339-353). A single enzyme subunit binds two Ca⁺⁺ ions and one heme b molecule (heme b (iron(II)-protoporphyrin IX) as cofactors which are essential for HRP's enzymatic activity.

Three synthetic open reading frames were designed to embed the enzymatic moiety of HRP into a frame work that ensure its expression on the surface of EVs.

Construct A (internal database entries 7516, 7518, 7520):

The first amino-terminal transmembrane domain of human CD63 (P08962; MLA1, TSPAN30; https://www.uniprot.org/uniprotkb/P08962/entry) encompassing aa 1-30 of CD63 (MAVEGGMKCVKFLLYVLLLAFCACAVGLIA; SEQ ID NO: 2) was fused to the enzymatic moiety of HRP, which was coupled to a flexible linker peptide (GGGGSGGGGGS; SEQ ID NO: 3) followed by the transmembrane domain and the cytoplasmic domain (LSMLVLQWASLAVLTLLLLLVMADCAFRRNLSTSHTYTTPPYDDAETYV; SEQ ID NO: 4) of the glycoprotein gp350 of Epstein-Barr virus (EBV) (https://www.uniprot.org/uniprotkb/P03200/entry).

The amino acid sequence of Construct A was reverse transcribed, codon-optimized and optimized for efficient translation following standard rules and algorithms. The deduced DNA nucleotide sequence was equipped with a close-to-perfect human Kozak consensus sequence, synthesized and molecularly cloned in E. coli using a standard vector plasmid.

The aa sequence of the complete synthetic protein reads (N-term to C-term):

(SEQ ID NO: 5)
MAVEGGMKCVKFLLYVLLLAFCACAVGLIAQLTPTFYDNSCPNVSNIVRD
TIVNELRSDPRIAASILRLHFHDCFVNGCDASILLDNTTSFRTEKDAFGN
ANSARGFPVIDRMKAAVESACPRTVSCADLLTIAAQQSVTLAGGPSWRVP
LGRRDSLQAFLDLANANLPAPFFTLPQLKDSFRNVGLNRSSDLVALSGGH
TFGKNQCRFIMDRLYNFSNTGLPDPTLNTTYLQTLRGLCPLNGNLSALVD
FDLRTPTIFDNKYYVNLEEQKGLIQSDQELFSSPNATDTIPLVRSFANST
QTFFNAFVEAMDRMGNITPLTGTQGQIRLNCRVVNSNSGGGGSGGGGSLS
MLVLQWASLAVLTLLLLLVMADCAFRRNLSTSHTYTTPPYDDAETYV

The DNA fragment was cloned into an appropriate expression plasmid (pcDNA3.1+) and, alternatively, into a basic retrovirus vector encoding the CD63:HRP:gp350 gene fusion, only.

### Construct B (Internal Database Entry 7522):

The CD63 sequence in Construct A was replaced by a conventional signal peptide (SP) (MQLLCVFCLVLLWEVGA; SEQ ID NO: 6) derived from the aa sequence of the glycoprotein gH of EBV (https://www.uniprot.org/uniprotkb/P03231/entry). The design of the synthetic fusion proteins mimics a type 1 transmembrane protein with its signal peptide. It is cleaved off by a SP peptidase when the protein transverses cellular membranes to be loaded, e.g., onto EVs or localizes to the cellular plasma membrane.

The aa sequence of the complete synthetic protein reads (N-term to C-term):

(SEQ ID NO: 7)
MQLLCVFCLVLLWEVGAQLTPTFYDNSCPNVSNIVRDTIVNELRSDPRIA
ASILRLHFHDCFVNGCDASILLDNTTSFRTEKDAFGNANSARGFPVIDRM
KAAVESACPRTVSCADLLTIAAQQSVTLAGGPSWRVPLGRRDSLQAFLDL
ANANLPAPFFTLPQLKDSFRNVGLNRSSDLVALSGGHTFGKNQCRFIMDR
LYNFSNTGLPDPTLNTTYLQTLRGLCPLNGNLSALVDFDLRTPTIFDNKY
YVNLEEQKGLIQSDQELFSSPNATDTIPLVRSFANSTQTFFNAFVEAMDR
MGNITPLTGTQGQIRLNCRVVNSNSGGGGSGGGGSLSMLVLQWASLAVLT
LLLLLVMADCAFRRNLSTSHTYTTPPYDDAETYV

The coding DNA fragment was cloned into an appropriate expression plasmid (pcDNA3.1+).

### Construct C (Internal Database Entry 7521):

The enzymatic moiety (aa 31-338) of HRP was molecularly cloned such that it became part of the frame work of the human CD63 protein, an integral membrane protein with four transmembrane domains. The largest extracellular moiety of CD63 in between transmembrane domain 3 and 4 was replaced by the enzymatic moiety of HRP flanked by flexible linker peptides similar to those in Construct A.

The aa sequence of the complete synthetic protein reads (N-term to C-term):

(SEQ ID NO: 8)
MAVEGGMKCVKFLLYVLLLAFCACAVGLIAVGVGAQLVLSQTIIQGATPG
SLLPVVIIAVGVFLFLVAFVGCCGACKENYCLMITFAIFLSLIMLVEVAA
AIAGYVFRDSGGGGGQLTPTFYDNSCPNVSNIVRDTIVNELRSDPRIAAS
ILRLHFHDCFVNGCDASILLDNTTSFRTEKDAFGNANSARGFPVIDRMKA
AVESACPRTVSCADLLTIAAQQSVTLAGGPSWRVPLGRRDSLQAFLDLAN
ANLPAPFFTLPQLKDSFRNVGLNRSSDLVALSGGHTFGKNQCRFIMDRLY
NFSNTGLPDPTLNTTYLQTLRGLCPLNGNLSALVDFDLRTPTIFDNKYYV
NLEEQKGLIQSDQELFSSPNATDTIPLVRSFANSTQTFFNAFVEAMDRMG
NITPLTGTQGQIRLNCRVVNSNSGGGGSGGGGSLRKNVLVVAAAALGIAF
VEVLGIVFACCLVKSIRSGYEVM

The coding DNA fragment was cloned into an appropriate expression plasmid (pcDNA3.1+).

### Expression of HRP Fusion Proteins in Cell Lines

Expression plasmids carrying Construct A, B or C were transiently transfected into HEK293 or 293T cells. One day prior to transfection, the cells were plated at a density of 3×10⁵ cells/ml in a 6-well plate. On the following day, transfection was performed by using TransIT-293 transfection reagent (Mirus Bio), according to the manufacturer's protocol. Per well, 2.5 μg of plasmid DNA were used.

Retroviral vectors encoding Construct A were packaged into a retroviral envelope following standard procedure (Engels et al., Hum Gene Ther (2003), 14(12): 1155-1168.) and the virus stocks were used to transduce a subline of HEK293 cells adapted to grow in suspension in synthetic cell culture medium (LV-MAX Production medium, Thermo Fisher). Briefly, 3×10⁶ HEK293 cells were mixed with 2 mL of virus supernatant containing 4 μg/mL protamine sulfate in a 6-well plate. Transduction was performed by spinoculation (centrifugation at 1000×g for 90 min, 32 °C). One week later, HRP-positive cells were sorted by using anti-mouse IgG MicroBeads (130-048-401, Miltenyi Biotec) with a mouse HRP-specific antibody (ab10183, Abcam), according to the manufacturer's protocol. Cell sorting was repeated until a HRP expression of more than 95% was reached, as determined by flow cytometry.

1. A method to determine the recovery rate of sample extracellular vesicles, the method comprising:
(a) mixing a biofluid sample comprising sample extracellular vesicles with a known amount of control extracellular vesicles, wherein the control extracellular vesicles comprise
a heterologous marker wherein said marker is located (i) on the outer side or (ii) on the inner side of the control extracellular vesicle and which is suitable for colorimetric, photometric, and/or fluorescent detection,
to obtain a mixture,
(b) isolating the extracellular vesicles from said mixture,
(c) detecting the amount of control extracellular vesicles among the isolated extracellular vesicles using
(c1) a colorimetric, photometric, or fluorescent detection assay, or
(c2) an immuno-based assay employing a binding agent which binds to said heterologous marker, wherein said marker is located on the outer side, and
(d) determining the recovery rate of said sample extracellular vesicles based on the ratio of the amount of control extracellular vesicles detected in step (c) after the isolation step to the known amount of control extracellular vesicles mixed with the biofluid sample prior to the isolation step.

2. The method according to claim 1, wherein said heterologous marker is a protein.

3. The method according to claim 1 or 2, wherein said control extracellular vesicles further comprise a membrane protein, and wherein said heterologous marker is connected to said membrane protein.

4. The method according to claim 3, wherein said heterologous marker is directly fused to said membrane protein, covalently linked to said membrane protein, or connected to said membrane protein via a linker.

5. The method according to claim 3 or 4, wherein said membrane protein directs its own release through vesicles as a membrane-bound protein.

6. The method according to any one of claims 3 to 5, wherein said membrane protein is selected from the group consisting of transmembrane proteins, Gi-coupled proteins, membrane-anchored receptors, tetraspanins, type I or II or III transmembrane proteins, and single or multipass transmembrane proteins.

7. The method according to any one of the preceding claims, wherein said heterologous marker is selected from the group consisting of peroxidase, alkaline phosphatase and beta-galactosidase.

8. The method according to any one of the preceding claims, wherein said heterologous marker is horseradish peroxidase (HRP).

9. The method according to any one of claims 1 to 8, wherein the amount of control extracellular vesicles detected in step (c) after the isolation step is quantitatively determined in step (d).

10. The method according to any one of claims 1 to 8, wherein the amount of control extracellular vesicles detected in step (c) after the isolation step is determined in step (d) at a predefined point of time after the isolation step.

11. The method according to any one of claims 1 to 8, wherein the amount of control extracellular vesicles detected in step (c) after the isolation step is determined in step (d) without lag time.

12. The method according to any one of the preceding claims, further comprising in step (c1) adding a fluorogenic or chromogenic substrate.

13. The method according to claim 12, wherein said substrate processed by the heterologous marker accumulates over time.

14. The method according to claim 12 or 13, wherein accumulation of the processed substrate is linear with time.

15. The method according to any one of claims 12 to 14, wherein the processed substrate can be measured at a discrete time point after the addition of the substrate.

16. The method according to any one of claims 12 to 14, wherein the amount of EV detected in step (c) is determined 5 min to 4 hours after the isolation step (b).

17. The method according to any one of the preceding claims, wherein said heterologous marker is HRP, and wherein said detection step (c) is carried out in the presence of a substrate of HRP, said substrate being a chromogenic substrate.

18. The method according to any one of the preceding claims, wherein said heterologous marker is HRP, and wherein said detection step (c) is carried out in the presence of a substrate of HRP, said substrate being selected from the group consisting of 2,2′-Azino-bis(3-ethylbenzthiazoline-6-sulfonis acid (ABTS), 3-amino-9-ethylcarbazole (AEC), 4-chlor-1-naphthol (CN), 3,3′-Diaminobenzidine (DAB), 3,3′,5,5′-Tetramethylbenzidine (TMB), o-Phenylenediamine (OPD), tyramine, homovanillic acid (HVA), and 4-Hydroxyphenylee acetic acid.

19. The method according to any one of the preceding claims, wherein said control extracellular vesicles further comprise
(iii) streptavidin or biotin
located on the outer side of the control extracellular vesicle.

20. The method according to any one of the preceding claims, wherein said biofluid sample is selected from the group consisting of plasma sample, serum sample, urine sample, saliva sample, tear drop sample, cerebrospinal fluid sample, spinal fluid sample, peritoneal sample, ascites sample, pleural sample, and joint fluid sample.

21. The method according to any one of the preceding claims, wherein said control extracellular vesicles further comprise a protein of interest whose presence or overexpression is considered to represent a medical condition.

22. The method according to claim 21, said method further comprising a step of detecting said protein of interest.

23. A kit comprising a control extracellular vesicle as defined in any one of the preceding claims.

24. The kit according to claim 23, further comprising a chromogenic or fluorogenic substrate.

25. The kit according to claim 24, wherein said substrate is selected from the group consisting of 2,2′-Azino-bis(3-ethylbenzthiazoline-6-sulfonis acid (ABTS), 3-amino-9-ethylcarbazole (AEC), 4-chloro-1-naphthol (4CN), 3,3′-Diaminobenzidine (DAB), 3,3′,5,5′-Tetramethylbenzidine (TMB), o-Phenylenediamine (OPD), tyramine, homovanillic acid (HVA), and 4-Hydroxyphenylee acetic acid.

26. The kit according to any one of claims 23 to 25, further comprising a binding agent which binds to said heterologous marker.

27. A method for preparing control extracellular vesicles according to any one of claims 1 to 22, comprising the steps
(i) introducing an expression system encoding said heterologous marker into a host cell,
(ii) culturing said cells in a medium, and
(iii) recovering released control extracellular vesicles from said medium.

28. The method according to claim 27, wherein in step (i) an expression system encoding a membrane protein as defined in any one of claims 3 to 6 is introduced into said host cell.

29. A method for preparing control extracellular vesicles according to any one of claims 1 to 22, comprising the steps
(i)′ adding a heterologous marker as defined in any one of claims 1 to 22 to extracellular vesicles to chemically conjugate said heterologous marker to said extracellular vesicles in a suitable medium, and
(ii)′ recovering control extracellular vesicles with conjugated heterologous markers from said medium.

30. The method according to claim 29, wherein said control extracellular vesicles further comprise a membrane protein as defined in any one of claims 3 to 6.

31. A method to calibrate a device for sample extracellular vesicles analysis, the method comprising:
(a) mixing a biofluid sample comprising sample extracellular vesicles with a known amount of control extracellular vesicles, wherein the control extracellular vesicles comprise:
a heterologous marker wherein said marker is located (i) on the outer side or (ii) on the inner side of the control extracellular vesicle and which is suitable for photometric, colorimetric, and/or fluorescent detection, to obtain a mixture,
(b) introducing said mixture into said device,
(c) analyzing a property of said control extracellular vesicles in said mixture,
(d) optionally analyzing said property again with one or more new combinations of device settings,
(e) deriving from said analysis of said control extracellular vesicles a combination of device settings which gives the exact property of said control extracellular vesicles used to obtain said mixture, and
(f) using said combination of device settings to calibrate the device against future subsequent analysis of samples containing extracellular vesicles with unknown properties, to determine the exact properties thereof.

32. A method to evaluate the isolation of extracellular vesicles, the method comprising:
(a) mixing a biofluid sample comprising sample extracellular vesicles with a known amount of control extracellular vesicles, wherein the control extracellular vesicles comprise:
a heterologous marker wherein said marker is located (i) on the outer side or (ii) on the inner side of the control extracellular vesicle and which is suitable for photometric, colorimetric, and/or fluorescent detection, to obtain a mixture,
(b) isolating the control extracellular vesicles from said mixture, and
(c) detecting the presence of the control extracellular vesicles isolated from said mixture.

33. The method of claim 31 or 32, further comprising in step (c) adding a fluorogenic or chromogenic substrate.


SEQUENCE LISTING (8 sequences)

SEQ ID NO: 1 - protein, 353 aa, Armoracia rusticana
MHFSSSSTLFTCITLIPLVCLILHASLSDAQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSLLHDMVEVVDFVSSM

SEQ ID NO: 2 - protein, 30 aa, Homo sapiens
MAVEGGMKCVKFLLYVLLLAFCACAVGLIA

SEQ ID NO: 3 - protein, 11 aa, synthetic construct
  REGION 1..11: note = linker sequence
GGGGSGGGGGS

SEQ ID NO: 4 - protein, 49 aa, Epstein Barr Virus
LSMLVLQWASLAVLTLLLLLVMADCAFRRNLSTSHTYTTPPYDDAETYV

SEQ ID NO: 5 - protein, 397 aa, synthetic construct
  REGION 1..397: note = Synthetic HRP-CD63-gp350 fusion protein
MAVEGGMKCVKFLLYVLLLAFCACAVGLIAQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSGGGGSGGGGSLSMLVLQWASLAVLTLLLLLVMADCAFRRNLSTSHTYTTPPYDDAETYV

SEQ ID NO: 6 - protein, 17 aa, Epstein Barr Virus
MQLLCVFCLVLLWEVGA

SEQ ID NO: 7 - protein, 384 aa, synthetic construct
  REGION 1..384: note = Synthetic HRP-CD63-gH fusion protein
MQLLCVFCLVLLWEVGAQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSGGGGSGGGGSLSMLVLQWASLAVLTLLLLLVMADCAFRRNLSTSHTYTTPPYDDAETYV

SEQ ID NO: 8 - protein, 473 aa, synthetic construct
  REGION 1..473: note = Synthetic HRP-CD63 fusion protein
MAVEGGMKCVKFLLYVLLLAFCACAVGLIAVGVGAQLVLSQTIIQGATPGSLLPVVIIAVGVFLFLVAFVGCCGACKENYCLMITFAIFLSLIMLVEVAAAIAGYVFRDSGGGGGQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNSGGGGSGGGGSLRKNVLVVAAAALGIAFVEVLGIVFACCLVKSIRSGYEVM